(19) United States
(12) Patent Application Publication
(10) Pub. No.: US 2003/0207789 A1
(43) Pub. Date: Nov. 6, 2003

(54) Isomer composition containing optically active ethyl trans-2,2,6-trimethylcyclohexylcarboxylate and fragrance composition containing the isomer composition

(75) Inventors: Takeshi Yamamoto, Hiratsuka-shi (JP); Shinya Watanabe, Hiratsuka-shi (JP); Hideo Ujihara, Hiratsuka-shi (JP); Toshimitsu Hagiwara, Hiratsuka-shi (JP)

Correspondence Address: FITZPATRICK CELLA HARPER & SCINTO, 30 ROCKEFELLER PLAZA, NEW YORK, NY, 10112, US

(73) Assignee: Takasago International Corporation, Ohta-ku (JP)

(21) Appl. No.: 10/390,752

(22) Filed: Mar. 19, 2003

(30) Foreign Application Priority Data
Mar. 22, 2002 (JP) P.2002-081954

(51) Int. Cl. A61K 7/46

(52) U.S. Cl. 512/22

(57) ABSTRACT

A geometrical isomer composition excellent in odor and in chemical resistance, which is applicable to a base material containing a strongly alkaline chemical or a strongly acidic chemical, which can suppress the deterioration with time and the color change of the base material in the case that the material is exposed to light, and which also have highly palatable and unique fruity floral fragrance. The composition is produced by mixing 93 to 99% by weight of optically active ethyl trans-2,2,6-trimethylcyclohexylcarboxylate represented by Formula-1 or a mixture thereof and 7 to 1% by weight of optically active ethyl cis-2,2,6-trimethylcyclohexylcarboxylate represented by Formula-2 or a mixture thereof, respectively.

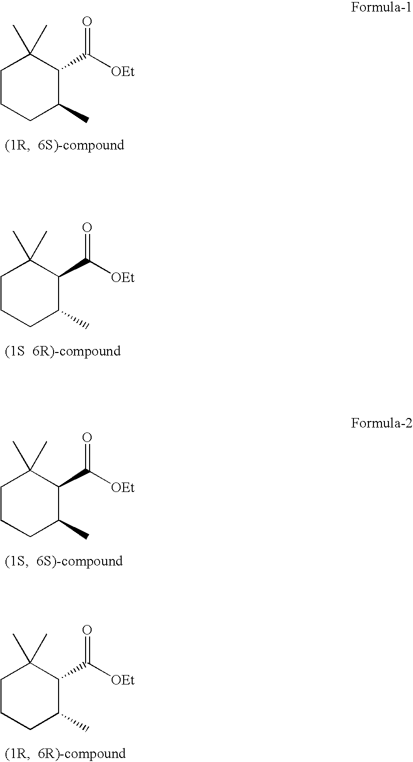

### FIELD OF THE INVENTION

The present invention relates to a geometrical isomer composition excellent in odor and in chemical resistance containing 93 to 99% by weight of optically active ethyl trans-2,2,6-trimethylcyclohexylcarboxylate or a mixture thereof and 7 to 1% by weight of optically active ethyl cis-2,2,6-trimethylcyclohexylcarboxylate or a mixture thereof, respectively, a process for producing the same, and a fragrance composition containing the same.

### BACKGROUND OF THE INVENTION

In recent years, it has been desired to develop fragrance materials having unique and superior fragrance characteristics owing to the increasing diversification of fragrant cosmetics, particularly the development of distinctive fruity floral fragrance materials has been highly requested.

During the development of new fragrance raw materials, the present inventors have found that the fragrance in an alkaline material such as soap is deteriorated with time and the color of the soap changes in the case that the material is exposed to light when the material is mixed with optically active ethyl 2,2,6-trimethylcyclohexylcarboxylate which is a fruity floral fragrance hitherto widely used. In the course of devising a measure to solve these problems, they have noticed that the fragrance in an alkaline material such as soap is hardly deteriorated with time and the color of the soap hardly changes in the case that the material is exposed to light when the material such as soap is mixed with a geometrical isomer composition containing about 98% by weight of optically active ethyl trans-2,2,6-trimethylcyclohexylcarboxylate {optical isomer ratio: (1R,6S)-isomer/(1S,6R)-isomer=99/1}.

Thus, an object of the invention is to provide a fragrance composition having unique fruity floral fragrance and exhibiting an excellent secondary physical properties such as stability to base materials and light stability. Particularly, it is to provide a geometrical isomer composition excellent in chemical resistance, which is applicable to a base material containing an alkaline chemical or an acidic chemical, which can suppress the deterioration with time and the color change of the base material in the case that the material is exposed to light, and which also have highly palatable and unique fruity floral fragrance. Furthermore, it is to provide a geometrical isomer composition excellent in chemical resistance, which is applicable to a base material containing an alkaline chemical or an acidic chemical, which can suppress the deterioration with time and the color change of the base material in the case that the material is exposed to light, and which also contains highly palatable ethyl 2,2,6-trimethylcyclohexylcarboxylate.

### SUMMARY OF THE INVENTION

For the purpose of solving the above problems, the inventors have studied on ethyl 2,2,6-trimethylcyclohexylcarboxylate including a question of cis/trans ratio. As a result, they have finally accomplished the invention.

Namely, they have found that a geometrical isomer composition containing 93 to 99% by weight of optically active ethyl trans-2,2,6-trimethylcyclohexylcarboxylate represented by Formula-1 or a mixture thereof and 7 to 1% by weight of optically active ethyl cis-2,2,6-trimethylcyclohexylcarboxylate represented by Formula-2 or a mixture thereof, respectively, is hardly hydrolyzed and exists stably under the conditions under which usual esters such as carboxylic acid esters having no α-substituent are easily hydrolyzed. Moreover, the composition is also completely stable to a strong acid such as hydrochloric acid.

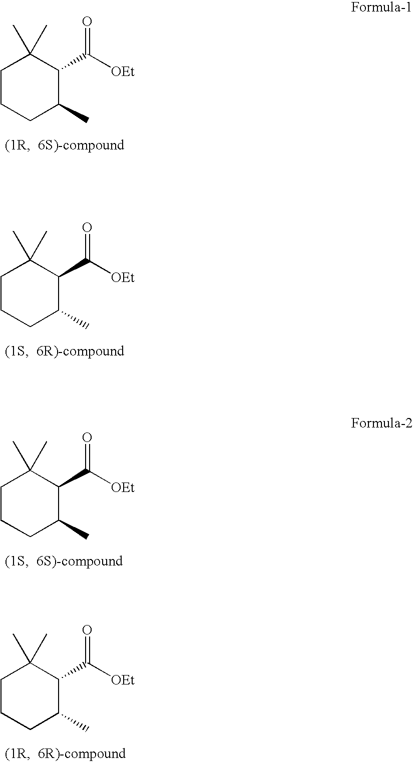

These facts entirely overthrow the common sense about the stability of fragrance compounds having a conventional ester group.

In this connection, it has been revealed that an isomer composition containing a geometrical isomer composition containing about 90% by weight or less of optically active ethyl trans-2,2,6-trimethylcyclohexylcarboxylate represented by Formula-1 or a mixture thereof and about 10% by weight or more of optically active ethyl cis-2,2,6-trimethylcyclohexylcarboxylate represented by Formula-2 or a mixture thereof is inferior in stability.

Furthermore, the inventors have found that a geometrical isomer composition of ethyl trans-carboxylate having a content of 93 to 99% by weight itself and a fragrance composition containing the same acquires totally excellent sensory evaluation and also can be widely used as a fragrance-improving agent in commercial goods in which a strongly basic compound such as ammonia or a very strongly basic compound such as sodium hydroxide or potassium hydroxide is used or commercial goods in which a strongly acidic substance such as hydrochloric acid is used.

Since ethyl (1R,6S)-2,2,6-trimethylcyclohexylcarboxylate which is an optically active trans-isomer is particularly excellent in fragrance quality and also excellent in chemical resistance, it has been revealed that a mixture containing a large amount of the above compound can be widely employed.

As a result, according to the invention, there are provided an isomer composition excellent in odor and in chemical resistance comprising a geometrical isomer composition containing 93 to 99% by weight of optically active ethyl trans-2,2,6-trimethylcyclohexylcarboxylate represented by Formula-1 or a mixture thereof and 7 to 1% by weight of optically active ethyl cis-2,2,6-trimethylcyclohexylcarboxylate represented by Formula-2 or a mixture thereof, respectively (hereinafter referred to as a geometrical isomer composition of ethyl trans-carboxylate having a content of 93 to 99% by weight), a fragrance composition containing the isomer composition, and a fragrance-scented product containing the fragrance composition.

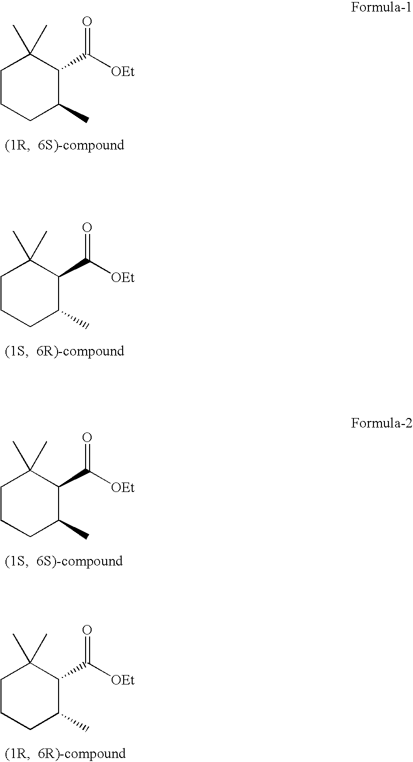

### DETAILED DESCRIPTION OF THE INVENTION

The following will explain the invention in detail.

The above isomer composition excellent in chemical resistance may be constituted by only the above geometrical isomer composition of ethyl trans-carboxylate having a content of 93 to 99% by weight or by the geometrical isomer composition and a blending agent commonly used in the fragrance technology field. In some cases, it is also possible to contain a compound produced as a by-product at the time when the geometrical isomer composition is produced.

As the above geometric composition, there may be mentioned a geometrical isomer composition containing 93 to 99% by weight of optically active ethyl trans-2,2,6-trimethylcyclohexylcarboxylate represented by Formula-1 {(1R,6S)-isomer and/or (1S,6R)-isomer} and 7 to 1% by weight of optically active ethyl cis-2,2,6-trimethylcyclohexylcarboxylate represented by Formula-2 {(1R,6R)-isomer and/or (1S,6S)-isomer} or a geometrical isomer composition containing 93 to 99% by weight of a mixture of equivalent amount of optically active ethyl trans-2,2,6-trimethylcyclohexylcarboxylate {(1S,6R)-isomer} and optically active ethyl trans-2,2,6-trimethylcyclohexylcarboxylate {(1R,6S)-isomer} (racemic form) and 7 to 1% by weight of a mixture of equivalent amount of optically active ethyl cis-2,2,6-trimethylcyclohexylcarboxylate {(1R,6R)-isomer} and optically active ethyl cis-2,2,6-trimethylcyclohexylcarboxylate {(1S,6S)-isomer} (racemic form), respectively.

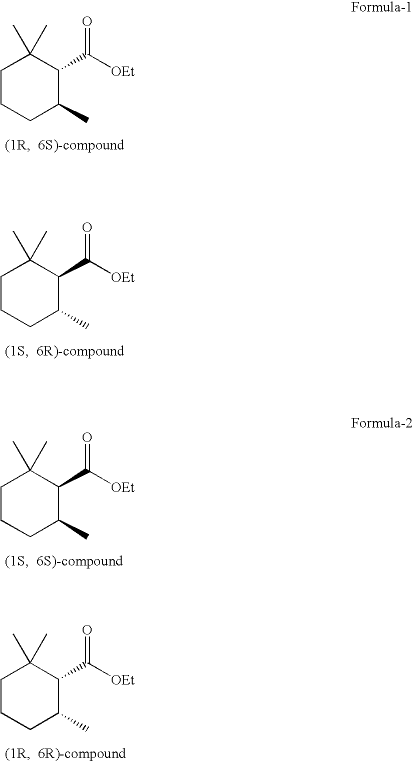

Moreover, the common blending agent to be added to the geometrical isomer includes glycols such as glycerin, diethylene glycol, and hexylene glycol; solvents used for common fragrances, such as diethyl phthalate and benzyl benzoate; and the like. It is possible to add basically any amount of the blending agent but the range of the amount to be used is usually from about 20 to 90% by weight on the basis of the geometrical isomer composition.

The following will explain the process for producing the isomer composition excellent in chemical resistance according to the invention.

As an isomer composition as a synthetic material for the above isomer composition excellent in chemical resistance, there may be mentioned an isomer composition containing a geometrical isomer composition containing 93 to 99% by weight of optically active trans-2,2,6-trimethylcyclohexyl methyl ketone represented by Formula-3 or a mixture thereof and 7 to 1% by weight of optically active cis-2,2,6-trimethylcyclohexyl methyl ketone represented by Formula-4 or a mixture thereof, respectively (hereinafter referred to as an isomer composition of trans-methyl ketone having a content of 93 to 99% by weight).

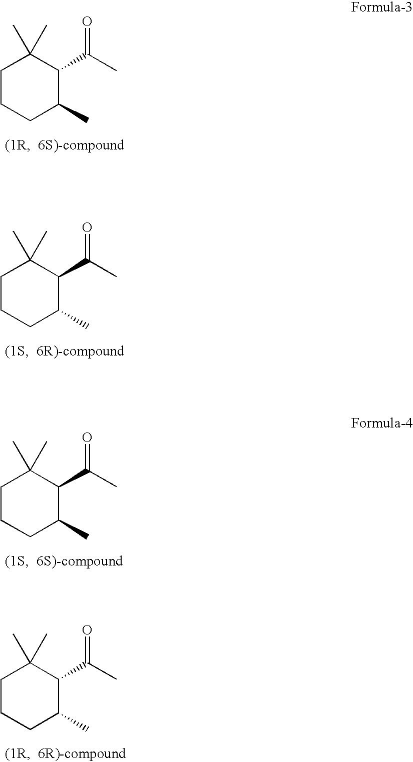

As examples of the geometrical isomer composition which is an essential constitutional component of the above isomer composition, there may be mentioned a geometrical isomer composition containing 93 to 99% by weight of optically active trans-2,2,6-trimethylcyclohexyl methyl ketone represented by Formula-3 {(1R,6S)-isomer and/or (1S,6R)-isomer} and 7 to 1% by weight of optically active cis-2,2,6-trimethylcyclohexyl methyl ketone represented by Formula-4 {(1R,6R)-isomer and/or (1S,6S)-isomer}, respectively, or a geometrical isomer composition containing 93 to 99% by weight of a mixture of equivalent amount of optically active trans-2,2,6-trimethylcyclohexyl methyl ketone {(1S,6R)-isomer} and optically active trans-2,2,6-trimethylcyclohexyl methyl ketone {(1R,6S)-isomer} (racemic form) and 7 to 1% by weight of a mixture of equivalent amount of optically active cis-2,2,6-trimethylcyclohexyl methyl ketone {(1R,6R)-isomer} and optically active cis-2,2,6-trimethylcyclohexyl methyl ketone {(1S,6S)-isomer} (racemic form), respectively.

The isomer composition according to the invention may be constituted by only the above geometrical isomer composition but, in some cases, it is possible to contain a compound produced as a by-product at the time when the geometrical isomer composition is produced.

In order to avoid complication, the process for producing the geometrical isomer composition which is an essential constitutional component for the isomer composition of trans-methyl ketone having a content of 93 to 99% by weight will be first explained in the following.

The above geometrical isomer composition can be produced by the method shown in JP-A-2001-348353.

Since no racemization occurs during the production process, the optical purity of the geometrical isomer composition to be prepared according to the above process reflects the optical purity of a starting material to be used.

For example, when (3S)-citronellal or (3R)-citronellal having a high optical purity {an optical purity of 98% e.e. (e.e.: enantiomer excess) in both cases} manufactured by Takasago International Corporation is used as a starting material, a product having a high optical purity (98% e.e.) is obtained. Namely, since the asymmetric carbon atom at the 3-position of the starting material (i.e., (3S) or (3R)) does not directly participate in the reaction and the carbon atom at the 3-position of the starting material corresponds to the carbon atom at the 6-position of the product, the optical purity of the asymmetric carbon atom at 6-position {i.e., (6S) or (6R)} of optically active trans-2,2,6-trimethylcyclohexyl methyl ketone which is a product reflects the optical purity of the asymmetric carbon atom at the 3-position of the starting material, and as a result, optically active trans-2,2,6-trimethylcyclohexyl methyl ketone having an optical purity of 98% e.e. is obtained.

In this connection, the geometrical isomer composition containing 93 to 99% by weight of racemic trans-2,2,6-trimethylcyclohexyl methyl ketone and 7 to 1% by weight of racemic cis-2,2,6-trimethylcyclohexyl methyl ketone, respectively, can be produced in accordance with the above process. Namely, by carrying out similar operations as in the above process using any one of racemic 7-methoxycitronellal, 7-hydroxycitronellal, and citronellal as a starting material, the geometrical isomer composition containing 93 to 99% by weight of racemic trans-2,2,6-trimethylcyclohexyl methyl ketone and 7 to 1% by weight of racemic cis-2,2,6-trimethylcyclohexyl methyl ketone, respectively, can be produced.

As a different production process, there may be mentioned a process wherein (1S,6R)-2,2,6-trimethylcyclohexyl methyl ketone which is an optically active trans-isomer and (1R,6S)-2,2,6-trimethylcyclohexyl methyl ketone which is another optically active trans-isomer are separately prepared beforehand and then both compounds are mixed each other to form a mixture of equivalent amount of the (1S,6R)-isomer and the (1R,6S)-isomer (racemic form).

A process based on Japanese Patent No. 2748184 may be mentioned as an alternative synthetic process for the geometrical isomer composition containing 93 to 99% by weight of optically active trans-2,2,6-trimethylcyclohexyl methyl ketone or a mixture thereof and 7 to 1% by weight of optically active cis-2,2,6-trimethylcyclohexyl methyl ketone or a mixture thereof, respectively.

Namely, the above composition can be produced using, as a starting material, optically active dihydrocyclocitral containing 90% by weight of its trans-isomer, which is a synthetic intermediate for the production of optically active 1-(2,2,6-trimethylcyclohexyl)-2-buten-1-one, or a racemic modification containing the dihydrocyclocitral.

More specifically, by reacting optically active dihydrocyclocitral (1) containing 90% by weight of its trans-isomer with methylmagnesium chloride to obtain a secondary alcohol (2) {the isomer composition ratio of cis-isomer to trans-isomer of the secondary alcohol (2) is the same as the composition of the starting material}, subjecting the secondary alcohol (2) to dehydrogenation in the presence of a copper-chromium catalyst, and carrying out distillation, an optically active trans-2,2,6-trimethylcyclohexyl methyl ketone composition having a trans-isomer content higher than that of the starting alcohol (2) (e.g., 93% by weight of the trans compound and 7% by weight of the cis compound) can be produced.

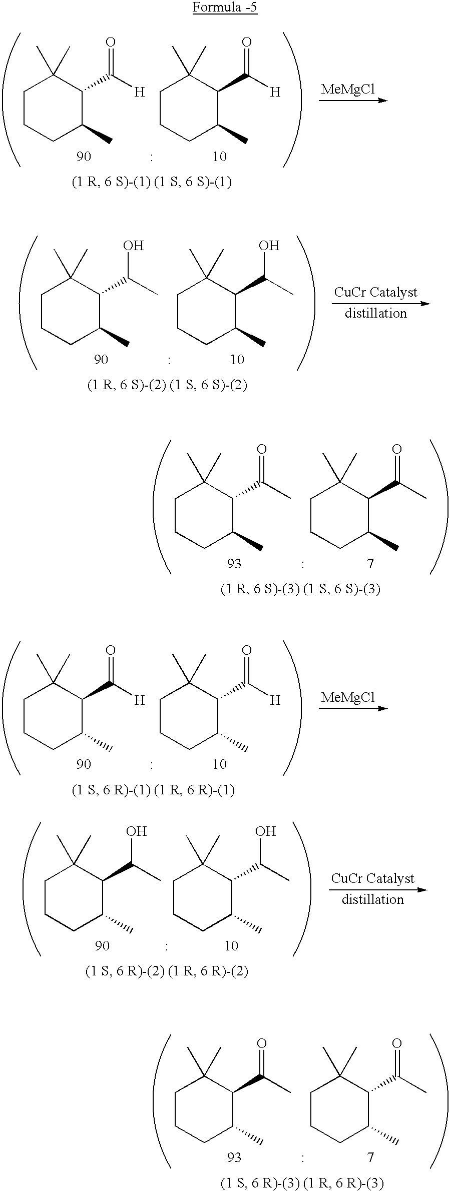

Using the thus synthesized geometrical isomer composition containing 93 to 99% by weight of optically active trans-2,2,6-trimethylcyclohexyl methyl ketone or a mixture thereof and 7 to 1% by weight of optically active cis-2,2,6-trimethylcyclohexyl methyl ketone or a mixture thereof, respectively, as a starting material, a geometrical isomer composition excellent in odor and in chemical resistance according to the invention containing 93 to 99% by weight of optically active ethyl trans-2,2,6-trimethylcyclohexylcarboxylate or a mixture thereof and 7 to 1% by weight of optically active ethyl cis-2,2,6-trimethylcyclohexylcarboxylate or a mixture thereof, respectively (hereinafter referred to as a geometrical isomer composition of ethyl trans-carboxylate having a content of 93 to 99% by weight which is excellent in odor and in chemical resistance) can be produced.

As representative examples of the above composition, there may be mentioned a geometrical isomer composition containing 93 to 99% by weight of optically active ethyl trans-2,2,6-trimethylcyclohexylcarboxylate {(1R,6S)-isomer and/or (1S,6R)-isomer} and 7 to 1% by weight of optically active ethyl cis-2,2,6-trimethylcyclohexylcarboxylate represented by Formula-2 {(1S,6S)-isomer and/or (1R,6R)-isomer}, respectively, or a geometrical isomer composition containing 93 to 99% by weight of a mixture of equivalent amount of optically active ethyl trans-2,2,6-trimethylcyclohexylcarboxylate {(1R,6S)-isomer} and optically active ethyl trans-2,2,6-trimethylcyclohexylcarboxylate {(1S,6R)-isomer} (racemic modification) and 7 to 1% by weight of a mixture of equivalent amount of optically active ethyl cis-2,2,6-trimethylcyclohexylcarboxylate {(1S,6S)-isomer} and optically active ethyl cis-2,2,6-trimethylcyclohexylcarboxylate {(1R,6R)-isomer} (racemic form).

The process for producing the above geometrical isomer composition of ethyl trans-carboxylate having a content of 93 to 99% by weight which is excellent in odor and in chemical resistance is explained by illustrating the case that a geometric isomer composition of optically active trans-2,2,6-trimethylcyclohexyl methyl ketone having trans/cis ratio of 98.6/1.4 {(1R,6S)-(1)/(1S,6S)-(1)=98.6/1.4} is used as a starting material.

Namely, as shown in Formula-6, 2,2,6-trimethylcyclohexylcarboxylic acid (4) is obtained by oxidizing the side chain methyl group of 2,2,6-trimethylcyclohexyl methyl ketone (3). The carboxylic acid (4) is a composition having the same geometrical isomer ratio as that of the starting ketone (3). Then, by converting the carboxylic acid (4) into its ethyl ester, aimed optically active ethyl 2,2,6-trimethylcyclohexylcarboxylate (5), i.e., a geometrical isomer composition having a slightly increased trans ratio {(1R,6S)-(5)/(1S,6S)-(5)=99/1) can be obtained.

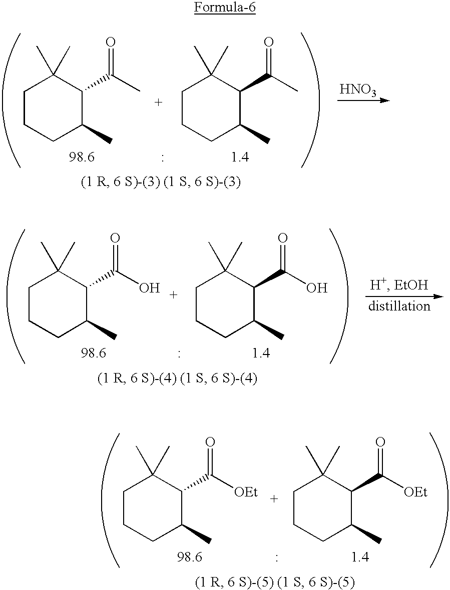

In this production process, the carboxylic acid (4) can be synthesized in good yields using a common oxidizing method such as oxidation with manganese dioxide, oxidation with nitric acid, oxidation with hypochloric acid, and oxidation with potassium permanganese as a oxidation method for use in the oxidation of the side chain methyl group of the geometrical isomer composition of the ketone (3).

With regard to the conversion into an ethyl ester in the final step, a general method can be usually employed. Namely, ethyl 2,2,6-trimethylcyclohexylcarboxylate (5) can be selectively produced by reacting a geometrical isomer composition of the carboxylic acid (4) with ethanol using a protonic acid such as sulfuric acid as a catalyst under dehydration and removal of water. Moreover, using an alkali metal salt such as sodium salt of the carboxylic acid (4) and ethyl halide or diethyl sulfate, ethyl 2,2,6-trimethylcyclohexylcarboxylate (5) can be also selectively produced.

The optical purity of the thus obtained geometrical isomer composition of ethyl trans-carboxylate having a content of 93 to 99% by weight means a numerical value calculated according to the following method. Namely, any one asymmetric carbon atom of optically active ethyl trans-2,2,6-trimethylcyclohexylcarboxylate is focused and an optical purity is determined by applying a usual method to the focused asymmetric carbon atom. For example, in the case of (1R,6S)-1-(2,2,6-trimethylcyclohexyl)-2-buten-1-one which is an optically active trans-isomer, the carbon atom at the 6-position is focused and, when 99% of the carbon atom is S-isomer and 1% thereof is R-isomer, the optical purity of the above (1R,6S)-1-(2,2,6-trimethylcyclohexyl)-2-buten-1-one is found to be 98%. In this connection, in the synthesis of ethyl 2,2,6-trimethylcyclohexylcarboxylate (5) as an aimed compound from the above starting ketone (1), since the optical purity of the starting ketone (1) is maintained, the optical purity of ethyl 2,2,6-trimethylcyclohexylcarboxylate (5) as an aimed compound reflects the optical purity of the above starting ketone (3).

The thus obtained geometrical isomer composition of ethyl trans-carboxylate having a content of 93 to 99% by weight is excellent in chemical resistance. Namely, the geometrical isomer composition is extremely stable even in a composition in which a strongly basic compound such as sodium hydroxide, potassium hydroxide, or ammonia (a pH value of about 10 or higher, further to about 14) or a strongly acidic substance such as hydrochloric acid (a pH value of about 3 or lower, further to 1) is present. The geometrical isomer composition excellent in chemical resistance defined by the invention means a geometrical isomer composition which is hardly deteriorated by the above strongly basic compounds and strongly acidic substances and can stably exist for a long period of time. More specifically, as described in Examples, it exhibits a good stability even in an alkaline base material such as soap, detergent, hairdye, or perm liquid, and is also stable under strongly acidic conditions such as hydrochloric acid. In the case that these base materials are scented with the above geometrical isomer composition, the deterioration of fragrance with time is hardly observed and an excellent and highly palatable fragrance can be provided. Moreover, as a result of scenting soap and testing its stability to light, it has been surprisingly revealed that the geometrical isomer composition of ethyl trans-carboxylate having a content of 93 to 99% by weight which is excellent in chemical resistance is hardly colored and satisfies a sufficient condition as fragrance for soap, while damascones (α-damascone, β-damascone, δ-damascone) are remarkably colored and fragrance thereof is also deteriorated.

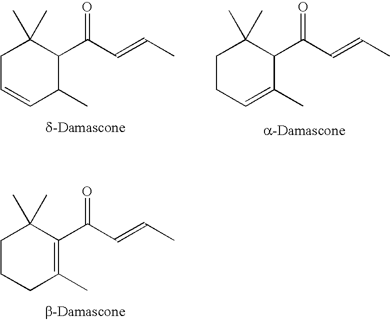

Furthermore, with regard to the secondary functional evaluations such as stability of the geometrical isomer composition containing 93 to 99% by weight of optically active ethyl trans-2,2,6-trimethylcyclohexylcarboxylate, as shown in Examples, since the composition is found to be stable under both of strongly acidic conditions (hydrochloric acid conditions) and strongly alkaline conditions (hydrolysis conditions for ester in sodium hydroxide/aqueous methanol system), it is suggested that commercial products in the region where an ester compound is hitherto supposed to be difficult to use are effectively scented and hence products having a highly palatable fragrance can be developed. As a result of application tests in commercial products, as shown in Examples, it has been revealed that the composition is very useful for masking a bad odor and scenting in commercial product groups including hypochlorite-based bleach using a very strong base such as sodium hydroxide, perm liquid and hairdye using a strong base such as ammonia, soap or detergent neutralized with an alkali such as sodium hydroxide, or toilet detergent or mold-removing liquid using a strong acid such as hydrochloric acid.

The thus obtained geometrical isomer composition of ethyl trans-carboxylate having a content of 93 to 99% by weight which is excellent in chemical resistance is a strongly diffusive, highly palatable, and unique fragrance-providing agent or fragrance-improving and enhancing agent. Also, as a fragrance component, it can be added to and blended with various fragrant cosmetics, health and hygiene materials, pharmaceuticals, and the like. Namely, product value can be enhanced by blending a suitable amount that is able to provide a unique fragrance to shampoo, body shampoo, rinse, perfume, cologne, hair tonic, hair cream, pomade, other cosmetic base materials for hair growth, face powder, lipstick, other cosmetic base materials and cosmetic cleaners, soap, liquid soap, dish washing soap, laundry detergent, softener, room air fresheners, furniture polish, hairdye, perm liquid, bleach, disinfectant, insecticide, repellent, other various types of health and hygiene cleaners, toothpaste, mouthwash, toilet paper and fragrances for facilitating the taking of pharmaceuticals. Of these, the composition is particularly effective for soap, liquid soap, hairdye, perm liquid, laundry detergent, softener, bleach, disinfectant, insecticide, other various types of health and hygiene cleaners, toothpaste, and mouthwash.

The geometrical isomer composition of ethyl trans-carboxylate having a content of 93 to 99% by weight which is excellent in odor and in chemical resistance can be solely added to and blended with the above commercial products, but it may be mixed with other fragrance or a conventional blending agent to prepare a new composition and then the composition may be added to the above products.

As the other fragrance, any fragrance can be employed without particular limitation. The conventional blending agent is also not particularly limited.

The blending amount of the above geometrical isomer composition or the composition containing the same in commercial products varies depending on the products and thus is difficult to determine sweepingly, but it can be commonly used in an amount of about 0.01 to 20% by weight, preferably 0.05 to 10% by weight.

The geometrical isomer composition containing 93 to 99% by weight of ethyl trans-2,2,6-trimethylcyclohexylcarboxylate or a mixture thereof provided by the invention is stable to base materials and light and also stable in an alkaline material or acidic material, and is useful as a floral fruity fragrance. Moreover, the geometrical isomer composition can be also provided at an inexpensive price. Furthermore, a fragrance composition using the geometrical isomer composition and a highly palatable fragrance-scented product using a strongly alkaline and/or strongly acidic material can be also provided.

The following will explain the invention in detail with reference to Examples, Test Examples, and Formulation Examples, but these Examples are described for the purpose of explanation only and the invention is not limited to these Examples.

### EXAMPLE-1

Synthesis of Geometrical Isomer Composition Containing 98.6% by Weight of Optically Active trans-2,2,6-trimethylcyclohexyl Methyl Ketone {Optical Isomer Ratio: (1R, 6S)-isomer/(1S,6R)-isomer=99/1}

1-1) Synthesis of (4S)-4,8-dimethyl-7-nonen-2-ol

Magnesium (50 g) and tetrahydrofuran (100 ml) were placed in a 2-liter, 4-necked flask equipped with an intake tube (for methyl chloride), thermometer, condenser and stirrer under a nitrogen stream, followed by heating to 40° C. and adding iodine (one piece) and methyl iodide (1 ml) to activate the magnesium. Then, tetrahydrofuran (600 ml) was added and methyl chloride gas was introduced through the intake tube under stirring. The resulting mixture was then allowed to react at 40 to 45° C. until the magnesium powder disappeared (requiring 2 to 3 hours) to prepare a solution of methylmagnesium chloride in tetrahydrofuran.

After completion of the reaction, the solution was cooled to room temperature and (3S)-citronellal (290 g: manufactured by Takasago International Corporation, optical purity: 98% e.e.) was added dropwise over a period of 3 hours under cooling with ice. After the dropwise addition, the solution was stirred at the same temperature for 2 hours. After the reaction was completed, the solution was cooled and a solution of water (108 g) in tetrahydrofuran (200 ml) was added dropwise to decompose the unreacted methyl magnesium chloride and the thus produced alkoxide. The formed magnesium chloride hydroxide as a solid was removed through a filter and the resulting solution of the product in tetrahydrofuran was concentrated with an evaporator to obtain a concentrated oil (316 g).

The resulting concentrated oil (316 g) was distilled with a Widmer distiller to obtain 296 g of (4S)-4,8-dimethyl-7-nonen-2-ol (b.p.: 70-71° C./133 Pa, [α]_{D}²⁴=+1.9° (c=1.02, EtOH)).

The purity of the resulting fraction of (4S)-4,8-dimethyl-7-nonen-2-ol on gas chromatography was found to be 99.8% by weight, and spectral data indicated the values shown below.

GC/MS (m/e); 170 (M⁺, 10%), 152 (2), 137 (8), 109 (70), 95 (65), 82 (100), 69 (80), 55 (50), 43 (76)

IR (NaCl); 3343 cm⁻¹ (br)

NMR [δ (CDCl₃)]; 5.10 (t, 1H, J=7.1 Hz), 3.89 (dq 1H, J=12.8, 6.2 Hz), 2.0 (m, 2H), 1.68 (s, 3H), 1.60 (s, 3H), 1.55 (m, 1H), 1.5 (m, 1H), 1.4 (m, 2H), 1.19 (d, 3H, J=6.2 Hz), 1.1 (m, 1H), 0.91 (d, 3H, J=6.6 Hz)

1-2) Synthesis of (4S)-4,8-dimethyl-7-nonen-2-one

Acetone (2,000 ml) and (4S)-4,8-dimethyl-7-nonen-2-ol (240 g) synthesized in Example 1-1 were placed in a 5-liter, 4-necked flask equipped with a dropping funnel, thermometer, condenser, and stirrer. Jones reagent {prepared from water (520 ml), concentrated sulfuric acid (165 g), and chromium trioxide (112 g)} placed in the dropping funnel was added dropwise over a period of 4 hours under cooling with ice. After the dropwise addition, the mixture was stirred for 2 hours. Then, sodium hydrogen sulfite was added gradually until the orange color of chromium (VI) disappeared. The liquid was then separated and the bottom layer was extracted with petroleum ether (1,000 ml). Since the liquid again separated into two layers when the extract was combined with the previous upper layer, the lower layer was added to the previous lower layer and further extracted three times with 500 ml of petroleum ether. After combining the petroleum ether layers and washing with a saturated brine, a saturated aqueous sodium hydrogen carbonate solution and again with a saturated brine, the solvent was evaporated and the resulting concentrated oil was distilled with a Widmer distiller to obtain 194 g of (4S)-4,8-dimethyl-7-nonen-2-one (b.p.: 63° C./133 Pa, [α]_{D}²⁴=−10.7° (c=1.00, EtOH)).

The purity of the resulting fraction of (4S)-4,8-dimethyl-7-nonen-2-ol on gas chromatography was found to be 99.5% by weight, and spectral data indicated the values shown below.

GC/MS (m/e); 168 (M⁺, 19%), 150 (8), 135 (25), 110 (58), 95 (100), 85 (42), 69 (47), 43 (64)

IR (NaCl); 1716 cm⁻¹

NMR [δ (CDCl₃)]; 5.09 (t, 1H, J=7.1 Hz), 2.42 (dd, 1H, J=5.6, 15.7 Hz), 2.22 (dd, 1H, J=8.2, 15.7 Hz), 2.0 (m, 1×3H), 1.68 (s, 3H), 1.60 (s, 3H), 1.3 (m, 1H), 1.2 (m, 1H), 0.91 (d, 3H, J=6.6 Hz)

1-3) Synthesis of (4S)-4,8-dimethyl-2,7-(and -1,7-)-nonadien-2-yl acetate

Under a nitrogen stream, (4S)-4,8-dimethyl-7-nonen-2-one (168 g) synthesized in Example 1-2, isopropenyl acetate (200 g), and p-toluenesulfonic acid monohydrate (19 g) were placed in a 3,000 ml, 4-necked flask equipped with a thermometer, condenser, and stirrer, followed by reaction at 90° C. for 22 hours under stirring. As a result of sampling the product and analyzing it by gas chromatography, it was confirmed by the mass spectral data shown below that three types of (4S)-4,8-dimethyl-2,7-(and 1,7)-nonadien-2-yl acetate: (4S)-(6) (area composition by gas chromatography: (4S)-(6-1), 50.3%; (4S)-(6-2), 16.4%; (4S)-(6-3) 26.8%} were formed.

The products were then subjected to a subsequent cyclization reaction without further treatment. Mass spectra of three types of (4S)-4,8-dimethyl-2,7-(and 1,7)-nonadien-2-yl acetate: (4S)-(6) (4S)-[6-1]; 210 (M⁺, 1%), 168 (17), 150 (68), 135 (38), 109 (77), 95 (57), 85 (100), 69 (47), 43 (100) (4S)-[6-2]; 210 (M⁺, 1%), 168 (10), 150 (57), 135 (30), 109 (68), 95 (43), 85 (98), 69 (36), 43 (100) (4S)-[6-3]; 210 (M+, 1%), 167 (8), 150 (64), 135 (38), 109 (100), 95 (75), 85 (30), 69 (62), 43 (94)

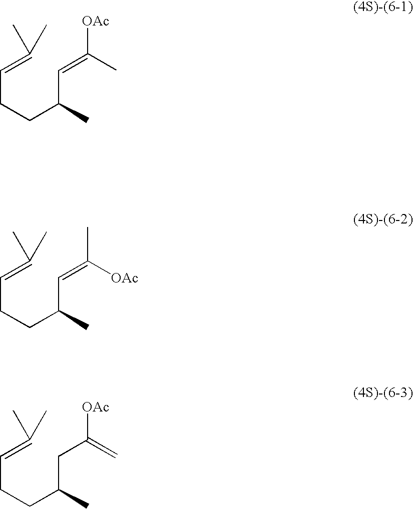

IR and NMR spectra of a mixture of three types of (4S)-4,8-dimethyl-2,7-(and 1,7)-nonadien-2-yl acetate: {(4S)-(6-1), (4S)-(6-2), and (4S)-(6-3)}

IR (NaCl); 1756 cm⁻¹, 1213 cm⁻¹

NMR [δ (CDCl₃)]; 5.06 (t, 1H), 4.74 (t, 1H, 10.6 Hz), 2.11 (s, 3H), 2.0 (m, 2H), 1.66 (s, 3H), 1.58 (s, 3H), 1.1 (m, 2H), 1.0 (m, 1H), 0.91 (d, 3H)

1-4) Synthesis of Geometrical Isomer Composition Containing 98.6% by Weight of Optically Active trans-2,2,6-trimethylcyclohexyl Methyl Ketone {Optical Isomer Ratio: (1R, 6S)-isomer/(1S,6R)-isomer=99/1}

85% by weight phosphoric acid (50 g) and toluene (1,500 ml) were added to a reaction liquid containing the three types of (4S)-4,8-dimethyl-2,7-(and 1,7)-nonadien-2-yl acetate {(4S)-(6-1), (4S)-(6-2), and (4S)-(6-3)} synthesized in Example 1-3 and the resulting mixture was reacted at 100° C. for 32 hours. After cooling the reaction liquid, the liquid was washed with water, a saturated aqueous sodium carbonate solution, and a saturated brine and then the solvent was evaporated. The resulting concentrated oil (area composition on gas chromatography: trans-isomer: 64.5%, cis-isomer: 1.1%, and small amounts of other unknown components: 34.4%) was purified with a 50-stage precision distiller to obtain 71 g of a geometrical isomer composition of optically active trans-2,2,6-trimethylcyclohexyl methyl ketone {optical isomer ratio: (1R, 6S)-isomer/(1S,6R)-isomer=99/1} (area ratio on gas chromatography: trans/cis=98.6/1.4, b.p.: 78° C./1197 Pa, [α]_{D}²⁴=−23.80° (c=1.01, EtOH)).

The resulting fraction of optically active trans-2,2,6-trimethylcyclohexyl methyl ketone demonstrated spectral data having the values shown below.

GC/MS (m/e); 168 (M⁺, 34%), 153 (10), 135 (17), 125 (20) 110 (62), 99 (100), 85 (45), 69 (89), 43 (62)

IR (NaCl); 1708 cm⁻¹

NMR [δ (CDCl₃)]; 2.16 (s, 3H), 2.07 (d, 1H, J=11.2 Hz), 1.8 (m, 1H), 1.7 (dq, 1H), 1.5 (m, 2H), 1.4 (ddd, 1H, J=1.4, 3.3, 13.1 Hz), 1.2 (m, 1H), 0.96 (s, 3H), 0.93 (s, 3H), 0.9 (m, 1H), 0.81 (d, 3H, J=6.3 Hz)

### EXAMPLE-2

Synthesis of Geometrical Isomer Composition Containing 99% by Weight of Racemic trans-2,2,6-trimethylcyclohexyl Methyl Ketone

Using racemic citronellal as a starting material, the reaction was carried out in the same manner as the synthetic operations in Example-1 to obtain 73 g of racemic trans-2,2,6-trimethylcyclohexyl methyl ketone (b.p.: 78° C./1197 Pa, area ratio on gas chromatography: trans/cis=99/1).

### EXAMPLE-3

Synthesis of Geometrical Isomer Composition Containing 93% by Weight of Optically Active trans-2,2,6-trimethylcyclohexyl Methyl Ketone {Optical Isomer Ratio: (1R,6S)-isomer/(1S,6R)-isomer=99/1}

3-1) Synthesis of Geometrical Isomer Composition Containing 90% by Weight of Optically Active trans-1-(2,2,6-trimethylcyclohexyl)ethan-1-ol {Optical Isomer Ratio: (1R,6S)-isomer/(1S,6R)-isomer=99/1)

Using a geometrical isomer composition (154 g) containing, as a main component, optically active trans-2,2,6-trimethylcyclohexylcarbaldehyde {optical isomer ratio: (1R,6S)-isomer/(1S,6R)-isomer=99/1} produced in accordance with the method of Example-2 of Japanese patent No. 2840899 (area ratio on gas chromatography: trans/cis=90/10), a reaction with methylmagnesium chloride was carried out in the same manner as the method described in Example 1-1 to obtain 146 g of a geometrical isomer composition of optically active trans-1-(2,2,6-trimethylcyclohexyl)ethan-1-ol {optical isomer ratio: (1R,6S)-isomer/(1S,6R)-isomer=99/1} (b.p.: 60° C./33.2 Pa, 24 [α]_{D}²⁴=−14.71° (c=1.01, EtOH), area ratio on gas chromatography: trans/cis=90/10).

The spectral data of the resulting geometrical isomer composition (146 g) indicated the values shown below.

GC/MS (m/e); 170 (M⁺, 0%), 152 (1), 137 (10), 125 (45), 111 (92), 95 (19), 83 (70), 69 (100), 55 (60), 41 (49)

IR (NaCl); 3442 cm⁻¹ (br)

NMR [δ (CDCl₃)]; 4.14 (q, 1H, J=6.9 Hz), 1.7 (m, 1H), 1.4 (m, 2H), 1.3 (m, 1H), 1.30 (d, 3H, 6.9 Hz), 1.2 (m, 1H), 1.15 (d, 3H, J=7.3 Hz), 1.1 (m, 1H), 1.0 (m, 1H), 0.91 (s, 3H), 0.90 (s, 3H), 0.81 (d, 1H, J=10.9 Hz)

3-2) Synthesis of Geometrical Isomer Composition Containing 93% by Weight of Optically Active trans-2,2,6-trimethylcyclohexyl Methyl Ketone {Optical Isomer Ratio: (1R,6S)-isomer/(1S,6R)-isomer=99/1}

The geometrical isomer composition (100 g) containing 90% by weight of optically active trans-1-(2,2,6-trimethylcyclohexyl)ethan-1-ol synthesized in Example 3-1 and a copper-chromium catalyst (5 g) pre-activated with hydrogen were placed in a 200 ml autoclave, followed by reaction at 200° C. for 6 hours. After cooling the reaction mixture and removing the catalyst by filtration, the reaction liquid was distilled with a Widmer distiller to obtain 89 g of a composition having an increased trans-isomer content by 3% by weight, i.e., a geometrical isomer composition containing 93% by weight of optically active trans-2,2,6-trimethylcyclohexyl methyl ketone {optical isomer ratio: (1R,6S)-isomer/(1S,6R)-isomer=99/1) {area ratio on gas chromatography: trans/cis=93/7, [α]_{D}²⁴=−21.92° (c=1.01, EtOH), b.p.: 76-78° C./1197 Pa)}.

### EXAMPLE-4

Synthesis of Geometrical Isomer Composition Containing 99% by Weight of Optically Active Ethyl trans-2,2,6-trimethylcyclohexylcarboxylate {Optical Isomer Ratio: (1R,6S)-isomer/(1S,6R)-isomer=99/1}

The geometrical isomer composition (50 g; 0.3 mol) containing 98.6% by weight of optically active trans-2,2,6-trimethylcyclohexyl methyl ketone obtained in Example 1 was added dropwise to 30% by weight aqueous nitric acid solution (400 ml) heated to 90° C. over a period of 5 minutes. Subsequently, the whole was stirred at the same temperature for 3 hours to complete the reaction. The product was treated in a usual manner to obtain 50.6 g of a geometrical isomer composition containing 98.6% by weight of optically active trans-2,2,6-trimethylcyclohexylcarboxylic acid {optical isomer ratio: (1R,6S)-isomer/(1S,6R)-isomer=99/1} {area ratio on gas chromatography: trans/cis=98.6/1.4; m.p.: 62-64° C., [α]_{D}²⁵=+13.670 (c=1.01, EtOH)}.

Then, Aliquat 336 (trade name) (5.3 g), potassium hydroxide powder (36.4 g), and a solution of the geometrical isomer composition containing 98.6% by weight of optically active trans-2,2,6-trimethylcyclohexylcarboxylic acid (45 g) in toluene (80 ml) were mixed each other, followed by 1 hour of stirring at 40° C. Thereafter, ethyl bromide (40 g) was added thereto at 40 to 45° C. for 1 hour. The whole was stirred at the same temperature for another 3 hours to complete the reaction. The product was treated in a usual manner and further purified with a Widmer distiller to obtain 37.6 g of a geometrical isomer composition containing 99.9% by weight of optically active ethyl trans-2,2,6-trimethylcyclohexylcarboxylate {optical isomer ratio: (1R,6S)-isomer/(1S,6R)-isomer=99/1, area ratio on gas chromatography: trans/cis=99/1, b.p.: 98° C./1333 Pa, [α]_{D}²⁵=+14.09° (c=1.01, EtOH)}.

### EXAMPLE-5

Synthesis of Geometrical Isomer Composition Containing 99% by Weight of Racemic Ethyl trans-2,2,6-trimethylcyclohexylcarboxylate

Using the geometrical isomer composition (50 g; 0.3 mol) containing 99% by weight of racemic trans-2,2,6-trimethylcyclohexyl methyl ketone obtained in Example 2, a reaction was carried out in the same manner as in Example-4 to obtain 36.5 g of a geometrical isomer composition containing 99.9% by weight or more of racemic ethyl trans-2,2,6-trimethylcyclohexylcarboxylate (area ratio on gas chromatography: trans/cis=99/1, b.p.: 98° C./1333 Pa).

### EXAMPLE-6

Synthesis of Geometrical Isomer Composition Containing 93% by Weight of Optically Active Ethyl trans-2,2,6-trimethylcyclohexylcarboxylate {Optical Isomer Ratio: (1R,6S)-isomer/(1S,6R)-isomer=99/1}

Using the geometrical isomer composition containing 93% by weight of optically active trans-2,2,6-trimethylcyclohexyl methyl ketone (optical isomer ratio: (1R,6S)-isomer/(1S,6R)-isomer=99/1} obtained in Example-3 instead of the geometrical isomer composition containing 98.6% by weight of optically active trans-2,2,6-trimethylcyclohexyl methyl ketone {optical isomer ratio: (1R,6S)-isomer/(1S,6R)-isomer=99/1} in Example-4, a reaction was carried out in the same manner as in Example-4 to obtain 37.2 g of a geometrical isomer composition containing optically active ethyl trans-2,2,6-trimethylcyclohexylcarboxylate {optical isomer ratio: (1R,6S)-isomer/(1S,6R)-isomer=99/1} (purity on gas chromatography: 99.9% or more, trans/cis area ratio=93/7, b.p.: 98° C./1333 Pa, [α]_{D}²⁵=+14.04° (c=1.01, EtOH)).

### COMPARATIVE EXAMPLE-1

Synthesis of Geometrical Isomer Composition Containing 90% by Weight of Optically Active Ethyl trans-2,2,6-trimethylcyclohexylcarboxylate {Optical Isomer Ratio: (1R,6S)-isomer/(1S,6R)-isomer=99/1}

In the process in Example 2 of Japanese Patent No. 2840899, a reaction was carried out in the same manner as in the above Example 2 with the exception that optically active 1-methoxycitronellal (manufactured by Takasago International Corporation; optical purity 98% e.e., [α]_{D}²⁵=−10.42°) was used instead of racemic methoxycitronellal, thereby 54 g of a geometrical isomer composition containing optically active ethyl trans-2,2,6-trimethylcyclohexylcarboxylate {optical isomer ratio: (1R,6S)-isomer/(1S,6R)-isomer=99/1} (purity on gas chromatography: 99.9% or more, trans/cis area ratio=90/10, b.p.: 98° C./1330 Pa, [α]_{D}²⁵=+14.01° (c=1.01, EtOH)} was obtained.

### COMPARATIVE EXAMPLE-2

Synthesis of Geometrical Isomer Composition Containing 90% by Weight of Racemic Ethyl trans-2,2,6-trimethylcyclohexylcarboxylate

A reaction was carried out in the same manner as in the process in Example 2 of Japanese Patent No. 2840899 to obtain 55 g of a geometrical isomer composition of racemic ethyl trans-2,2,6-trimethylcyclohexylcarboxylate {b.p.: 98° C./1333 Pa, purity on gas chromatography: 99.9% or more, and trans/cis area ratio=90/10}.

### COMPARATIVE EXAMPLE-3

Synthesis of Geometrical Isomer Composition of Racemic Ethyl trans-2,2,6-trimethylcyclohexylcarboxylate (trans/cis=32/68)

A reaction was carried out in the same manner as in the process in Example 1 of Japanese Patent No. 2840899 to obtain 55 g of a geometrical isomer composition of racemic ethyl trans-2,2,6-trimethylcyclohexylcarboxylate {b.p.: 86-90° C./1064 Pa, purity on gas chromatography: 99.9% or more, and trans/cis area ratio=32/68}.

### TEST EXAMPLE-1

Hydrolysis Stability Test of Geometrical Isomer Composition Containing 99% by Weight of Optically Active Ethyl trans-2,2,6-trimethylcyclohexylcarboxylate {Optical Isomer Ratio: (1R,6S)-isomer/(1S,6R)-isomer=99/1}

A methanol solution containing the geometrical isomer composition containing 99% by weight of the optically active compound (5) synthesized in Example 4 was prepared according to the following formulation.

Hydrolysis stability test of the solution was carried out under the following conditions.

| Formulation | |
|---|---|
| Geometrical isomer composition of the | 3.0 g |
| compound (5) synthesized in Example 4 | |
| Sodium hydroxide | 1.2 g |
| Methanol | 6.0 g |
| Water | 6.0 g |
| 1-Hexanol (internal standard) | 1.2 g |

Test conditions; The above solution was kept at methanol-refluxing temperature for 2 hours. As another test, the solution was kept at methanol-refluxing temperature for 4 hours.

After the above solution was kept for 2 hours or 4 hours, the amount of ethyl trans-2,2,6-trimethylcyclohexylcarboxylate was measured by internal standard method on gas chromatography. As a result, it was revealed that 99.9% by weight or more of the compound remained unreacted in both cases. Namely, it was confirmed that ethyl trans-2,2,6-trimethylcyclohexylcarboxylate completely stably existed surprisingly even under very strongly alkaline conditions.

### TEST EXAMPLE-2

Stability Test of Geometrical Isomer Composition Containing 99% by Weight of Racemic Ethyl trans-2,2,6-trimethylcyclohexylcarboxylate in Aqueous Hydrochloric Acid Solution

A methanol-hydrochloric acid solution containing the geometrical isomer composition of the racemic compound (5) synthesized in Example 5 was prepared according to the following formulation.

| Formulation | weight |
|---|---|
| 10% by weight aqueous | 1 g |
| hydrochloric acid solution | |
| Methanol | 10 g |
| Geometrical isomer composition of the | 0.1 g |
| compound (5) synthesized in Example 5 | |
| 1-Dodecane (internal standard) | 0.01 g |

The solution was placed in an Erlenmeyer flask and was stirred at 40° C. for 24 hours. The reaction mixture was treated in a usual manner and analyzed on gas chromatography. As a result, it was revealed that racemic ethyl trans-2,2,6-trimethylcyclohexylcarboxylate was not decomposed at all.

### TEST EXAMPLE-3

Composition Ratio of Trans-Isomer/Cis-Isomer and Scenting and Stability Test of Ethyl 2,2,6-trimethylcyclohexylcarboxylate in Soap

In the present test, using a soap base material (sodium salt of a mixture of 20% by weight of palm and 80% by weight of beef tallow) which was one of representative base materials, six kinds of scented soaps were obtained by scenting the soap base material with 1% by weight of six kinds of the geometrical isomer compositions of ethyl 2,2,6-trimethylcyclohexylcarboxylate (5) synthesized in Examples and Comparative Examples and also a blank soap was prepared. The scented soaps were each placed in a glass vessel, which was then tightly closed. After the vessel was stored in a constant temperature chamber at 40° C. for a certain period of time (1 month or 3 months), change of the fragrance was investigated by three expert panelists. As another test, change of suntan when the soaps were exposed to sunlight (after 1 month) was tested on six kinds of the 1% by weight-scented soaps and a blank soap prepared similarly.

The results are shown in Table-1.

In this connection, the symbols in the table are as follows.

TABLE 1
Results of scenting and stability test on
geometrical isomer composition of compound (5) in soap

| | | | Fragrance | | Suntan |
|---|---|---|---|---|---|
| | | | quality | | After |
| | | | After | After | 2 months |
| Experi- | Geometrical isomer compo- | | 1 month | 3 months | at room |
| ment | sition of compound (5) | trans/cis | at 50° C. | at 50° C. | temperature |
| 1 | Example-4 | 99/1 | ⊚ | ⊚ | ⊚ |
| | (1R, 6S)/(1S, 6S) = | | | | |
| 2 | Example-5 | 99/1 | ⊚ | ⊚ | ⊚ |
| | Racemic modification | | | | |
| 3 | Example-6 | 93/7 | ⊚ | ◯ | ⊚ |
| | (1S, 6R)/(1R, 6R) = | | | | |
| 4 | Comparative Example-1 | 90/10 | ◯ | Δ | Δ |
| | (1R, 6S)/(1S, 6S) = | | | | |
| 5 | Comparative Example-2 | 90/10 | ◯ | Δ | Δ |
| | Racemic modification | | | | |
| 6 | Comparative Example-3 | 32/68 | Δ | X | X |
| | Racemic modification | | | | |

Fragrance quality
⊚ absolutely no change
◯ almost no change
Δ presence of slight decomposition smell
X presence of decomposition smell
Suntan
⊚ the same as blank
◯ about the same as blank
Δ slightly colored
X colored

As a result, as shown in Experiment Nos. 4 to 6, the compounds containing 90% by weight or less of trans-isomer exhibited values which might cause some problems in fragrance stability and light stability, but as shown in experiment Nos. 1 to 3, the compositions containing 93% by weight of its trans-isomer were found to be excellent in fragrance stability and light stability.

### TEST EXAMPLE 4

Stability Test of Geometric Composition of Ethyl 2,2,6-trimethylcyclohexylcarboxylate (5) in Hypochlorite-Based Liquid Bleach Composition

According to the following formula, a liquid bleach composition scented with 0.1% of a geometrical isomer composition of the compound (5). As the geometrical isomer composition of the compound (5), six kinds of samples synthesized in Examples and Comparative Examples were investigated as shown in Table 2.

| Formulation | weight part |
|---|---|
| Sodium hypochlorite | 4.0 |
| Sodium dodecyl ether sulfate | 2.0 |
| (2 mol adduct of ethylene oxide) | |
| Sodium 2-ethylhexyl sulfate | 2.0 |
| Sodium hydroxide | 1.0 |
| Compound (5) synthesized in | 0.1 |
| each Example or Comparative Example | |
| Water | balance |
| Total | 100.0 |

Table 2 shows the test results of stability of the geometrical isomer compositions of the compound (5) in the sodium hypochlorite-based liquid bleach composition. As first test item, in order to investigate whether bleaching activity was secondarily deteriorated by the compound of the invention, each of six kinds of the above bleach compositions and a blank sample containing no geometrical isomer composition of the compound (5) was placed in a plastic bottle, which was then tightly closed. The bottles were placed in a constant temperature chamber and stored at 40° C. for 30 days. The amount of active hypochlorite of each stored samples was investigated by titration.

Then, masking ability against chlorine smell of the sodium hypochlorite-based liquid bleach composition by the geometrical isomer composition of the compound (5) was investigated. Each of the above six kinds of bleach compositions and blank sample was stored at 40° C. for 30 days and fragrance change of each stored sample was evaluated by three expert panelists.

The results are shown in Table-2.

TABLE 2
Test results of stability of geometrical isomer
composition of the compound (5) in sodium hypochlorite-
based liquid bleach composition

| Experi- | Geometrical isomer | | | | Change of |
|---|---|---|---|---|---|
| ment | composition of | trans/ | | | fragrance |
| No. | compound (5) | cis | (A) | (B)** | quality** |
| 1 | Example-4 | 99/1 | the same | ⊚ | None |
| | (1R, 6S)/(1S, 6S) = | | as blank | | |
| 2 | Example-5 | 99/1 | the same | ⊚ | None |
| | Racemic modification | | as blank | | |
| 3 | Example-6 | 93/7 | the same | ⊚ | (1) |
| | (1R, 6S)/(1S, 6S) = | | as blank | | |
| 4 | Comparative Example-1 | 90/10 | 2% | ⊚ | (2) |
| | (1R, 6S)/(1S, 6S) = | | decrease* | | |
| 5 | Comparative Example-2 | 90/10 | 2% | ◯ | (2) |
| | Racemic modification | | decrease* | | |
| 6 | Comparative Example-3 | 32/68 | 5% | Δ | (3) |
| | Racemic modification | | decrease* | | |

(A) Decrease of bleaching activity (%)
(B) Masking ability against chlorine smell
*decrease % of the activity when the activity of the blank sample is regarded as 100
**⊚; very good, ◯; good, Δ; moderate
(1) Decomposition smell was hardly observed,
(2) Decomposition smell was slightly observed.
(3) Decomposition smell was observed.

As a result, the samples of Experiment Nos. 1 to 3 exhibited the same values as that of the blank sample and decrease of the activity by the fragrances was not observed at all. In Experimental Nos. 4 to 6, decrease of the activity by the fragrances was observed although the decrease was 5% or less. With regard to masking of the chlorine smell, the samples except the sample of Experiment No. 6 were evaluated to be excellent in masking the chlorine smell. Furthermore, with regard to fragrance change after 30 days of the storage, no decomposition smell was observed in Experiment Nos. 1 to 3.

From theses test results, it was revealed that the fragrances of Experiment Nos. 1 to 3, i.e., the geometrical isomer compositions containing 93 to 99% by weight of ethyl trans-2,2,6-trimethylcyclohexylcarboxylate were excellent in scenting and masking for sodium hypochlorite-based liquid bleach composition.

### TEST EXAMPLE 5

Stability and Masking Test of Geometric Composition of Ethyl 2,2,6-trimethylcyclohexylcarboxylate (5) in Cold Perm Liquid

Five kinds of scented cold two-bath type perm liquid with the following formulation containing thioglycolic acid as a main component were prepared using the geometric compositions of the compound (5) synthesized in Examples and Comparative Examples.

| Formulation of first agent | |
|---|---|
| of cold perm liquid | weight part |
| Ammonium thioglycolic acid | 10.0 |
| (50% by weight aqueous solution) | |
| Aqueous ammonia | 1.5 |
| POE(20) oleyl ether (NIKKOL BO-20) | 1.0 |
| Compound (5) synthesized in each | 0.2 |
| Example and Comparative Example | 0.2 |
| Propylene glycol | 5 |
| Sodium edetate | 0.1 |
| Purified water | 82.2 |
| Total | 100% by weight |

With regard to the five kinds of scented cold two-bath type cold perm liquid, degree of masking ammonia smell and total sensory evaluation was investigated by five expert panelists. As a result, all the five panelists indicated the results shown in Table-3 and made an evaluation that the geometrical isomer compositions of the compound (5) having a trans-isomer ratio of 93 to 99% in Experiment Nos. 1 to 3 were excellent in masking the smell.

TABLE 3
Test results of masking and stability of
geometrical isomer composition of the compound (5) when
used in cold perm liquid

| Experi- | | | | | | |
|---|---|---|---|---|---|---|
| ment | Geometrical isomer compo- | | (A) | | (B) | |
| No. | sition of compound (5) | trans/cis | (C) | (D) | (C) | (D) |
| 1 | Example-4 | 99/1 | (1) | ◯ | (2) | ◯ |
| | (1R, 6S)/(1S, 6S) = | | | | | |
| 2 | Example-5 | 99/1 | (1) | ◯ | (2) | ◯ |
| | Racemic modification | | | | | |
| 3 | Example-6 | 93/7 | (1) | ◯ | (3) | ◯/Δ |
| | (1R, 6S)/(1S, 6S) = | | | | | |
| 4 | Comparative Example-1 | 90/10 | (1) | ◯ | (4) | Δ/X |
| | (1R, 6S)/(1S, 6S) = | | | | | |
| 5 | Comparative Example-2 | 90/10 | (1) | ◯ | (4) | Δ/X |
| | Racemic modification | | | | | |

(A) Test at the time when scented cold perm liquid of the geometrical isomer composition of the compound (5) was prepared
(B) Test after the passage of 2 months at 40° C. from the time when scented cold perm liquid of the geometrical isomer composition of the compound (5) was prepared
(C) Masking of ammonia smell
(D) Total evaluation
Notes)
⊚; very good, ◯; good, Δ; moderate, X; no good
(1) almost masked
(2) no change in fragrance tone
(3) almost no change in fragrance tone
(4) slight change in fragrance tone

### TEST EXAMPLE 6

Stability and Masking Test of Geometric Composition of Ethyl 2,2,6-trimethylcyclohexylcarboxylate (5) in Hairdye Liquid

Five kinds of scented hairdye first liquids with the following formulation were prepared using the geometric compositions of the compound (5) synthesized in Examples and Comparative Examples.

| Formulation of hairdye first liquid | weight part |
|---|---|
| p-Phenylenediamine | 2.0 |
| Aqueous ammonia | 5.0 |
| Resorcin | 1.0 |
| Polyoxyethylene cetyl ether | 3.0 |
| Cetanol | 7.0 |
| Liquid paraffin | 2.0 |
| Sodium sulfite | 0.3 |
| Sodium edetate | 0.3 |
| Compound (5) synthesized in each | 0.2 |
| Example and Comparative Example | |
| Purified water | 79.2 |
| Total | 100 |

With regard to the five kinds of the scented hairdye first liquids, degree of masking ammonia smell and total sensory evaluation was investigated by three expert panelists. As a result, good results were obtained as shown in Table-4 at the time when the scented hairdye first liquids were prepared. However, in the test after 1 month of storage at 50° C., the geometrical isomer compositions of the compound (5) having a trans-isomer composition ratio of 99% in Experiment Nos. 1 and 2 were evaluated to be excellent in masking the smell, while masking results were poor in Experiment Nos. 3 and 4 in which the trans-isomer composition ratio was 93% or less.

TABLE 4
Test results of masking and stability of
geometrical isomer composition of the compound (5) when
used in hairdye first liquid

| Experi- | | | | | | |
|---|---|---|---|---|---|---|
| ment | Geometrical isomer compo- | | (A) | | (B) | |
| No. | sition of compound (5) | trans/cis | (C) | (D) | (C) | (D) |
| 1 | Example-4 | 99/1 | (1) | ⊚ | (2) | ◯ |
| | (1R, 6S)/(1S, 6S) = | | | | | |
| 2 | Example-5 | 99/1 | (1) | ◯ | (2) | ◯ |
| | Racemic modification | | | | | |
| 3 | Example-6 | 93/7 | (1) | ◯ | (3) | ◯/Δ |
| | (1R, 6S)/(1S, 6S) = | | | | | |
| 4 | Comparative Example-1 | 90/10 | (1) | ◯ | (4) | Δ/X |
| | (1R, 6S)/(1S, 6S) = | | | | | |
| 5 | Comparative Example-2 | 90/10 | (1) | ◯/Δ | (4) | X |
| | Racemic modification | | | | | |

(A) Test at the time when the liquid was prepared
(B) Test after 1 month of storage at 50° C.
(C) Masking of ammonia smell
(D) Total evaluation
Notes)
⊚; very good, ◯; good, Δ; moderate, X; no good
(1) almost masked
(2) no change in fragrance tone
(3) almost no change in fragrance tone
(4) slight change in fragrance tone

### TEST EXAMPLE-7

Room Air Refresher Using Ethyl 2,2,6-trimethylcyclohexylcarboxylate (5)

Highly palatable rose-like fragrances with the following formulation was prepared using the geometrical isomer compositions of the compound (5) synthesized in Examples and Comparative Examples.

| Formulation | weight part |
|---|---|
| Compound (5) synthesized in each | 100 |
| Example and Comparative Example | |
| Geraniol | 110 |
| Nonylaldehyde | 2 |
| β-Ionone | 30 |
| cis-3-Hexenyl acetate | 3 |
| l-Citronellol | 80 |
| l-Citronellyl acetate | 7 |
| Phenylethylaldehyde dimethyl acetal | 50 |
| Isocyclocitral | 6 |
| 1-Rose oxide | 10 |
| cis-3-Hexenol | 7 |
| Methyl eugenol | 20 |
| Nerol | 20 |
| Phenylethyl acetate | 65 |
| Phenylethyl alcohol | 450 |
| Dihydrodamascone | 15 |
| p-t-Butyldihydrocinnamaldehyde | 25 |
| Total | 1000 |

Using these fragrance compositions, room air refreshers with the following formulation was prepared. Each of the air refreshers was placed in a 50-liter odorless box, which was then tightly closed. The box was allowed to stand at room temperature for 3 hours to diffuse fragrance. Thereafter, intensity of the odor of the air in the box and palatability were investigated by three expert panelists. Also, after used at room temperature for 1 month, quality and intensity of the fragrance were tested similarly.

| Formulation | weight part |
|---|---|
| Carrageenan | 2.0 |
| Roast bean gum | 0.2 |
| Calcium chloride | 0.2 |
| Ion-exchange water | 84 |
| Emulsifier | 0.3 |
| Glycol | 7.0 |
| BHT | 0.3 |
| Fragrance composition prepared | 6.0 |
| with the above formulation | |
| Total | 100.0 |

As a result, as shown in Table-5, in the test after 1 month use, the intensity slightly decreased commonly, but it was revealed that the air refreshers wherein ethyl 2,2,6-trimethylcyclohexylcarboxylate having a trans-isomer composition of 93 to 99% by weight (No. 1 to No. 3) was used could overcome the problems of fragrance change and long-term persistence of fragrance (important factors for commercialization).

TABLE 5
Results of sensory test in the case that
geometrical isomer composition of the compound (5) was used
as air refresher

| Experi- | | | (A) | | (B) | |
|---|---|---|---|---|---|---|
| ment | Geometrical isomer compo- | | Fra- | Total | Fra- | Total |
| No. | sition of compound (5) | trans/cis | grance | evaluation | grance | evaluation |
| 1 | Example-4 | 99/1 | (1) | ⊚ | (4) | ◯ |
| | (1R, 6S)/(1S, 6S) = | | | | | |
| 2 | Example 5 | 99/1 | (2) | ◯ | (4) | ◯ |
| | Racemic modification | | | | | |
| 3 | Example-6 | 93/7 | (1) | ⊚ | (4) | ◯ |
| | (1R, 6S)/(1S, 6S) = | | | | | |
| 4 | Comparative Example-1 | 90/10 | (1) | ◯ | (5) | Δ |
| | (1R, 6S)/(1S, 6S) = | | | | | |
| 6 | Comparative Example-2 | 90/10 | (3) | ◯/Δ | (5) | Δ |
| | Racemic modification | | | | | |

(A) Test at the time when the air refresher was prepared
(B) Test after the air refresher was used for 1 month
Notes)
(1) strongly diffusive and excellent rose floral note
(2) diffusive and excellent rose floral note
(3) diffusive and rose floral note
(4) almost no change of fragrance tone and slightly weak intensity
(5) slight change of fragrance tone and weaker intensity than that of No. 4
⊚; very good, ◯; good, Δ; moderate

### TEST EXAMPLE-8

Toilet Detergent Using Ethyl 2,2,6-trimethylcyclohexylcarboxylate (5)

A citrus floral fragrance composition was prepared using the geometrical isomer composition containing 99% by weight of ethyl trans-2,2,6-trimethylcyclohexylcarboxylate {optical isomer ratio; (1R,6S)-isomer/(1S,6R)-isomer=99/1} synthesized in Example-4 for an acidic detergent for toilet having citrus floral fragrance tone. The composition was added to a fragrance-free acidic detergent for toilet in an amount of 0.2% by weight. The mixture was placed in a plastic vessel, which was tightly closed and stored at room temperature for 90 days. Then, storage stability was investigated.

| Formulation | weight part |
|---|---|
| Geometrical isomer composition of | 500 |
| Compound (5) synthesized in Example 4 | |
| Cinnamic alcohol | 160 |
| 2,6-Dimethylheptan-2-ol | 200 |
| 1-Methylfenchyl alcohol | 2 |
| Orange terpene | 100 |
| Methyl salicylate | 38 |
| Total | 1000 |

As a result of judgment by three expert panelists, no change in fresh citrus floral fragrance tone was observed. Also, as a result of washing a dirty toilet, the washing effect was the same as in the case of a fragrance-free blank sample. Furthermore, in the case of the fragrance-added one, the whole toilet was filled with fresh and comfortable citrus floral fragrance after washing the toilet.

### TEST EXAMPLE-9

Fragrance Composition for Mold-Removing Liquid Using Ethyl 2,2,6-trimethylcyclohexylcarboxylate

A fragrance composition for mold-removing liquid having lilac floral fragrance was prepared according to the following formulation. The fragrance composition was added to a chlorate-based mold-removing liquid in an amount of 0.2% by weight. The mixture was placed in a plastic vessel, which was tightly closed and stored at 40° C. for 60 days. Then, storage stability was investigated. As a result of judgment by three expert panelists, no change in highly palatable lilac floral fragrance tone was observed. Also, the washing effect was the same as in the case of a

| Formulation | weight part |
|---|---|
| Geometrical isomer composition of | 230 |
| Compound (5) synthesized in Example 5 | |
| p-Cresyl methyl ketone | 20 |
| 2,6-Dimethylheptan-2-ol | 100 |
| 1-Methylfenchyl alcohol | 20 |
| Cedryl methyl ether | 30 |
| 3,7-Dimethyloctan-3-yl ethyl ether | 600 |
| Total | 1000 |

While the invention has been described in detail and with reference to specific embodiments thereof, it will be apparent to one skilled in the art that various changes and modifications can be made therein without departing from the scope thereof.

This application is based on Japanese patent application No. 2002-81954 filed Mar. 22, 2002, the entire contents thereof being hereby incorporated by reference.

What is claimed is:

1. A process for producing an isomer composition excellent in odor and in chemical resistance comprising a geometrical isomer composition containing 93 to 99% by weight of optically active ethyl trans-2,2,6-trimethylcyclohexylcarboxylate represented by Formula-1 or a mixture thereof and 7 to 1% by weight of optically active ethyl cis-2,2,6-trimethylcyclohexylcarboxylate represented by Formula-2 or a mixture thereof, which comprises oxidizing a side chain methyl group of a geometrical isomer composition containing 93 to 99% by weight of optically active trans-2,2,6-trimethylcyclohexyl methyl ketone or a mixture thereof and 7 to 1% by weight of optically active cis-2,2,6-trimethylcyclohexyl methyl ketone or a mixture thereof to obtain a geometrical isomer composition containing 93 to 99% by weight of optically active trans-2,2,6-trimethylcyclohexylcarboxylic acid or a mixture thereof and 7 to 1% by weight of optically active cis-2,2,6-trimethylcyclohexylcarboxylic acid or a mixture thereof and subsequently converting the optically active trans-2,2,6-trimethylcyclohexylcarboxylic acid and/or optically active cis-2,2,6-trimethylcyclohexylcarboxylic acid in the geometrical isomer composition into their ethyl esters, respectively: 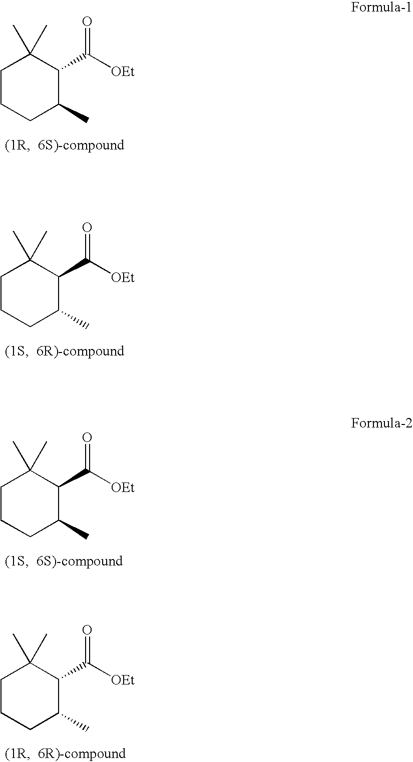
(wherein Et represents an ethyl group).

2. The process for producing a geometrical isomer composition excellent in chemical resistance according to claim 1, wherein the mixture thereof in the optically active ethyl trans-2,2,6-trimethylcyclohexylcarboxylate or mixture thereof is a racemic form of optically active ethyl trans-2,2,6-trimethylcyclohexylcarboxylate represented by Formula-1 and the mixture thereof in the optically active ethyl cis-2,2,6-trimethylcyclohexylcarboxylate or mixture thereof is a racemic form of optically active ethyl cis-2,2,6-trimethylcyclohexylcarboxylate represented by Formula-2:

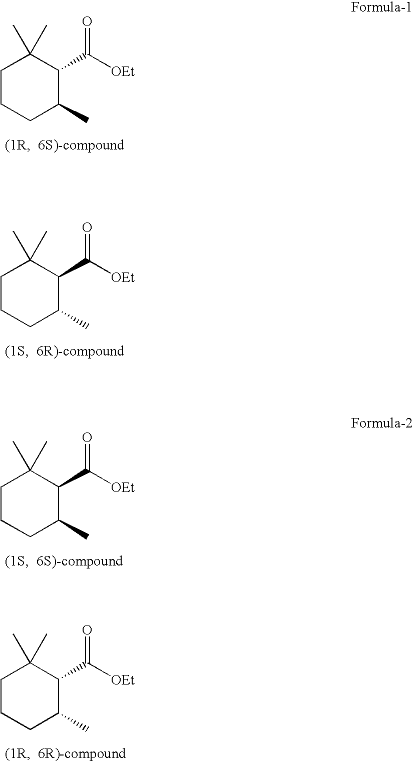

3. An isomer composition excellent in odor and in chemical resistance comprising a geometrical isomer composition containing 93 to 99% by weight of optically active ethyl trans-2,2,6-trimethylcyclohexylcarboxylate represented by Formula-1 or a mixture thereof and 7 to 1% by weight of optically active ethyl cis-2,2,6-trimethylcyclohexylcarboxylate represented by Formula-2 or a mixture thereof, respectively:

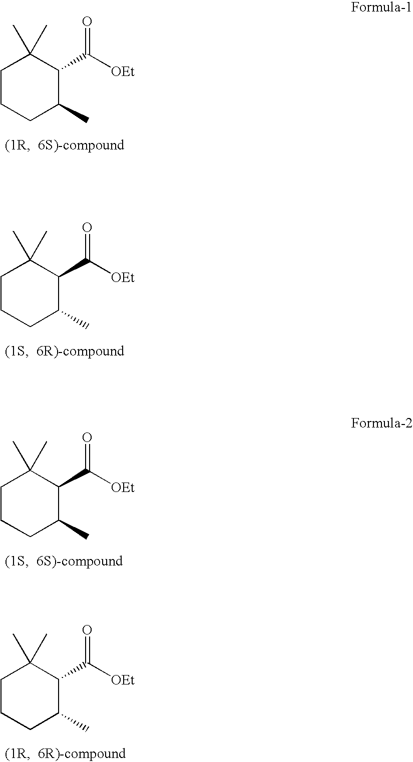

4. The isomer composition excellent in odor and in chemical resistance according to claim 3, wherein the mixture thereof in the optically active ethyl trans-2,2,6-trimethylcyclohexylcarboxylate or mixture thereof is a racemic form of optically active ethyl trans-2,2,6-trimethylcyclohexylcarboxylate represented by Formula-1 and the mixture thereof in the optically active ethyl cis-2,2,6-trimethylcyclohexylcarboxylate or mixture thereof is a racemic form of optically active ethyl cis-2,2,6-trimethylcyclohexylcarboxylate represented by Formula-2:

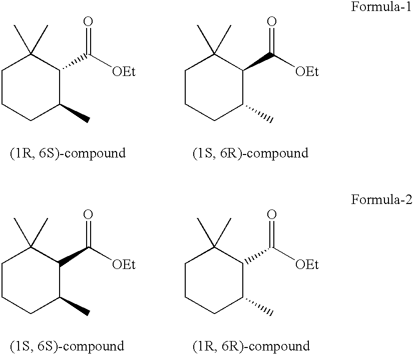

5. A fragrance composition containing the isomer composition excellent in odor and in chemical resistance according to claim 3 or 4 as an effective component.

6. A fragrance-scented product containing the fragrance composition according to claim 5.
